(19) United States
(12) Patent Application Publication
(10) Pub. No.: US 2024/0060097 A1
(43) Pub. Date: Feb. 22, 2024

(54) BIOCONVERSION OF FERULIC ACID TO VANILLIN

(71) Applicant: BASF SE, Ludwigshafen am Rhein (DE)

(72) Inventors: Nicholas A. LYONS, Cambridge, MA (US); Rui ZHOU, Acton, MA (US); David NUNN, Lowell, MA (US)

(21) Appl. No.: 18/267,478

(22) PCT Filed: Dec. 17, 2021
(86) PCT No.: PCT/US2021/064129

§ 371 Date: Jun. 15, 2023

Related U.S. Application Data
(60) Provisional application No. 63/127,449, filed on Dec. 18, 2020.

(51) Int. Cl. C12P 7/22; C12N 1/20; C07K 14/195; C12N 15/01

(52) U.S. Cl. CPC C12P 7/22; C12N 1/20; C07K 14/195; C12N 15/01

(57) ABSTRACT

A chemical mutagenic approach and a screening protocol were followed to select mutant strains of Amycolatopsis sp. with the ability to produce natural vanillin at high yields without any lag period. The resulting mutant strains are free of any exogeneous genetic elements and can be qualified as non-genetically modified organisms (non-GMOs) for regulatory purposes.

### CROSS REFERENCE TO RELATED APPLICATIONS

This application claims the benefit of U.S. Provisional Patent Application No. 63/127,449, filed Dec. 18, 2020, which is imported herein by reference in its entirety.

### SEQUENCE LISTING

The instant application contains a Sequence Listing that has been submitted electronically in ASCII format and is hereby incorporated by reference in its entirety. Said ASCII copy, created on Dec. 17, 2021, is named 074008_2038_WO_000324_SL.txt and is 11,734 bytes in size.

### FIELD OF THE INVENTION

The present disclosure generally relates to microorganisms and bioconversion processes for the fermentative production of natural vanillin using ferulic acid as the feedstock. The microorganisms useful in the present invention may be genetically engineered without introducing any exogenous nucleic acid sequences into the microbial cells. Hence the microorganisms of the present invention useful in the bioconversion of ferulic acid to vanillin are non-genetically modified organisms (non-GMOs).

### BACKGROUND OF THE INVENTION

Vanilla flavors are among the most frequently used flavors worldwide. They are used in the flavorings of numerous foods such as ice cream, dairy products, desserts, confectionary, bakery products and spirits. They are also used in perfumes, pharmaceuticals, and personal hygiene products.

Natural vanilla flavor has been obtained traditionally from the fermented pods of vanilla orchids (Vanilla planifolia). It is formed mainly after the harvest during several weeks of a drying and fermentation process of the beans by hydrolysis of vanillin glucoside that is present in the beans. The essential aromatic substance of vanilla flavor is vanillin (4-hydroxy 3-methoxybenzaldehyde).

About 12,000 tons of vanillin are consumed annually, of which only 20-50 tons are extracted from vanilla beans, the rest is produced synthetically, mostly from petrochemical-derived guaiacol. In recent years, there is a growing interest in producing vanillin through biological fermentation using renewable feedstocks such as ferulic acid derived from rice bran, coniferyl alcohol from Spruce tree lignin, corn sugar and eugenol from clove oil. Vanillin derived from renewable feedstocks using biological fermentation is recognized as “natural vanillin” by regulatory and legislative authorities and can be marketed as “natural products”.

The actinomycete Amycolatopsis sp. strain ATCC 39116 has been in use for the bioconversion of ferulic acid to vanillin. This microorganism is known to metabolize ferulic acid using four major pathways distinguished by the initial reaction, namely nonoxidative decarboxylation, side chain reduction, coenzyme A-independent deacetylation, and coenzyme A-dependent deacetylation. The coenzyme A-dependent deacetylation pathway for ferulic acid metabolism within the Amycolatopsis sp. strain ATCC 39116 includes two steps. In the first step, ferulic acid is subjected to non-oxidative deacetylation to yield vanillin (FIG. 1). This step is mediated by two enzymes, namely, feruloyl-coenzyme A (CoA) synthetase encoded by the fcs gene, and enoyl-CoA hydratase/aldolase encoded by the ech gene. Both genes (ech and fcs) are within a single operon and the expression of these two genes is suppressed when Amycolatopsis sp. is grown in a culture medium containing glucose as the source of carbon. It is only after the addition of ferulic acid that the transcription of these genes is induced. As a result, when ferulic acid is added to the culture medium, there is a lag period of about 5 hours or more before vanillin synthesis could be detected. Typically, the lag period is at least 4 hours and as many as 8 hours. Once vanillin starts accumulating, the second stage of ferulic acid metabolism kicks in. In the second step, vanillin is subjected to β-oxidation to produce vanillic acid. The conversion of vanillin to vanillic acid is mediated by vanillin dehydrogenase enzyme coded by the vdh gene. To increase vanillin production, there is a need for bioengineering such that the bioconversion of ferulic acid to vanillin starts without any lag period and the conversion of vanillin to vanillic acid is blocked or significantly reduced.

### SUMMARY OF THE INVENTION

The present invention provides a novel method for genetically engineering Amycolatopsis sp. to increase vanillin production from ferulic acid. The Amycolatopsis sp. genetically engineered according to the present invention do not harbor any exogenous nucleic acid sequences and can be qualified as non-genetically modified organisms (non-GMO).

The present invention provides a bioconversion process for producing vanillin using ferulic acid as a feedstock. The microorganism of the present invention is grown initially in a glucose-containing growth medium and the fermentative production of vanillin was initiated by means of adding ferulic acid to the culture medium. In a preferred embodiment of the present invention, the microorganism can be of the order Actinomycetales and the genus Amycolatopsis. For example, the microorganism can be an Amycolatopsis sp. strain accessible under number ATCC 39116. Unlike its wild-type counterpart, the mutant strains of the present invention can initiate the fermentative production of vanillin from ferulic acid without any lag period. The present strains can be obtained using chemical mutagenesis and the nature of the genetic mutations conferring the phenotype of producing vanillin using ferulic acid without any lag period can be identified by using whole genome sequencing.

The bioconversion of ferulic acid to vanillin in Amycolatopsis is carried out by two enzymes; namely, enoyl—coA hydratase/aldolase encoded by the ech gene and feruloyl-coenzyme A (CoA) synthetase encoded by the fcs gene. The ech and fcs genes in Amycolatopsis are within a single operon (ech-fcs operon) and controlled by a single promoter. When Amycolatopsis is grown in glucose-containing medium, the transcription of the ech-fcs operon is repressed. When ferulic acid is added, the repression of the ech-fcs operon is relieved although it takes several hours before the bioconversion of ferulic acid to vanillin is detectable.

In one aspect of the present invention, spores of Amycolatopsis sp. ATCC 39116 are subjected to chemical mutagenesis (e.g., using methyl methanesulfonate) and the resulting colonies can be screened to select a mutant strain in which the ech-fcs operon has overcome the repression. Using whole genome sequencing, the inventors unexpectedly found that the mutant strains with the desired phenotype of vanillin production without a lag period comprise one or more mutations in an endogenous gene comprising the nucleotide sequence of SEQ ID NO: 4 (which the inventors have named the echR gene due to the fact that it encodes a repressor of the ech-fcs operon). This EchR protein comprises the amino acid sequence of SEQ ID NO: 5 and was found to contain a MarR-type transcriptional repressor domain. According to the present invention, mutant strains according to the present invention can include one or more mutations in the echR gene selected from the group consisting of a deletion, an insertion, a frameshift mutation, a promoter mutation, a missense mutation, a nonsense mutation, a slicing mutation, a point mutation, and combinations thereof, where such mutation(s) is capable of causing functional inactivation of the EchR protein leading to the de-repression of the ech-fcs operon.

In another aspect, the present invention relates to the effect of the one or more mutations in the echR gene leading to the functional inactivation of the EchR protein in vanillin production using ferulic acid as a feedstock. In one embodiment, the present invention relates to a mutant strain of Amycolatopsis sp. with a functionally inactive echR gene capable of producing at least 0.5 g of vanillin per liter of culture medium within 5 hours after ferulic acid is initially fed to the mutant strain. In another embodiment of the present invention, the mutant strain of Amycolatopsis sp. with functionally inactive echR gene can produce at least 0.75 g of vanillin per liter of culture medium within 5 hours after ferulic acid is initially fed to the mutant strain. In yet another embodiment of the present invention, the mutant strain of Amycolatopsis sp. with functionally inactive echR gene can produce at least 0.5 g of vanillin per liter of culture medium within 3 hours after ferulic acid is initially fed to the mutant strain.

In an embodiment of the present invention, the mutant strain of Amycolatopsis sp. having one or more mutations in the endogenous echR gene comprising the nucleic acid sequence of SEQ ID NO: 4 produces at least 100% more vanillin when compared to a wild-type strain having no mutations in the endogenous echR gene, within 8 hours after ferulic acid is fed to the mutant strain or the wild-type strain. In another embodiment of the present invention, the mutant strain of Amycolatopsis sp. having a mutation in the endogenous echR gene having a nucleic acid sequence as in SEQ ID NO: 4 produces at least 200% more vanillin when compared to a wild-type strain having no mutation in the endogenous echR gene, within 8 hours after ferulic acid is fed to the mutant strain or the wild-type strain. In yet another embodiment of the present invention, the mutant strain of Amycolatopsis sp. having a mutation in the endogenous echR gene having a nucleic acid sequence as in SEQ ID NO: 4 produces at least 300% more vanillin when compared to a wild-type strain having no mutation in the endogenous echR gene, within 8 hours after ferulic acid is fed to the mutant strain or the wild-type strain. In another embodiment of the present invention, the mutant strain of Amycolatopsis sp. having a mutation in the endogenous echR gene having a nucleic acid sequence as in SEQ ID NO: 4 produces at least 400% more vanillin when compared to a wild-type strain having no mutation in the endogenous echR gene, within 8 hours after ferulic acid is fed to the mutant strain or the wild-type strain.

In another embodiment, the present invention provides a method for preventing the catabolism of the vanillin produced using ferulic acid within a strain of Amycolatopsis sp. having a mutation in the endogenous echR gene. Once vanillin is produced within Amycolatopsis sp. cells, it is converted to vanillic acid by vanillin dehydrogenase (Vdh) encoded by the vdh gene. In one aspect of the present invention, the vdh gene is subjected to one or more marker-less genetic modifications; for example, by using CRISPR technology or any other suitable recombinant DNA technology known to one skilled in the art, so that the vanillin dehydrogenase gene is no more functional within the Amycolatopsis sp. cell. The functional inactivation of the vanillin dehydrogenase can be achieved by mutating the vdh gene. According to the present invention, any one of the mutations in the vdh gene selected from the group consisting of a deletion, an insertion, a frameshift mutation, a promoter mutation, a missense mutation, a nonsense mutation, a slicing mutation and a point mutation that is capable of causing functional inactivation of vanillin dehydrogenase will prevent the conversion of vanillin into vanillic acid.

In one aspect, the present disclosure relates to a method of producing vanillin using an isolated genetically engineered Amycolatopsis sp. strain with a mutation in the echR gene. Further, the present Amycolatopsis sp. strain can be characterized by having no exogenous nucleic acid molecules and therefore can be qualified as a non-genetically modified organism (non-GMO). In some embodiments, the present bioconversion process of producing vanillin from ferulic acid can include (i) cultivating the isolated non-GMO host cell in a medium; (ii) adding ferulic acid to the medium to begin the bioconversion of ferulic acid to vanillin; and (iii) extracting vanillin from the medium.

The bioconversion method described herein can include recovering the vanillin from the mixture. The recovery of vanillin can be performed according to any conventional isolation or purification methodology known in the art. Prior to the recovery of the vanillin, the method also can include removing the biomass (enzymes, cell materials, etc.) from the fermentation mixture.

Vanillin produced using the methods and/or the isolated recombinant host cells described herein can be collected, purified, and incorporated into a number of consumable products. For example, the vanillin can be admixed with the consumable product. In some embodiments, the vanillin can be incorporated into the consumable product in an amount sufficient to impart, modify, boost or enhance a desirable taste, flavor, or sensation, or to conceal, modify, or minimize an undesirable taste, flavor or sensation, in the consumable product. The consumable product, for example, can be selected from the group consisting of food, food ingredients, food additives, beverages, drugs and tobacco. The consumable product, for example, can be selected from the group consisting of fragrances, cosmetics, toiletries, home and body care, detergents, repellents, fertilizers, air fresheners, and soaps.

A first embodiment is a non-GMO mutant strain of Amycolaptosis sp., wherein said strain is capable of producing at least 0.5 g vanillin per liter medium within 5 hours after ferulic acid is initially fed to the mutant strain, in this embodiment the strain is non-naturally occurring, in some embodiments the strain is a non-naturally occurring strain.

A second embodiment is a strain of the first embodiment, wherein said mutant strain is capable of producing at least 0.75 g vanillin per liter medium within 5 hours after ferulic acid is initially fed to the mutant strain in this embodiment the strain is non-naturally occurring.

A third embodiment is a strain of the first embodiment, wherein said mutant strain is capable of producing at least 0.5 g vanillin per liter medium within 3 hours after ferulic acid is initially fed to the strain in this embodiment the strain is non-naturally occurring.

A fourth embodiment is a strain of the first through the third embodiments, wherein said mutant strain comprising a mutation in an endogenous gene having the nucleic acid sequence of SEQ ID NO: 4. wherein the mutation reduces or eliminates suppression of ech-fcs operon.

A fifth embodiment is a strain of the first through the fourth embodiments, wherein the strain encodes an endogenous protein having a mutation in the amino acid sequence of SEQ ID. NO: 5.

A sixth embodiment is a mutant strain of the first through the fifth embodiments, further including a mutation in the vdh gene having a nucleic acid sequence of SEQ ID. NO: 6.

A seventh embodiment is a mutant strain of the fourth embodiment, wherein said mutation is a deletion, an insertion, a frameshift mutation, a promoter mutation, a missense mutation, a nonsense mutation, a slicing mutation or a point mutation.

An eighth embodiment is a mutant strain of the seventh embodiment, wherein the mutation is a deletion.

A nineth embodiment is a mutant strain of the seventh embodiment, wherein the mutation is a frameshift mutation.

A tenth embodiment is a mutant strain of the seventh embodiment, wherein the mutation is a promoter mutation.

An eleventh embodiment is a strain of first through the tenth embodiments, wherein the strain is a mutant of the strain Amycolatopsis sp. accessible under number ATCC 39116.

A twelfth embodiment is a mutant strain of the first through the eleventh embodiments, wherein the strain produces at least 100% more vanillin when compared to a wild-type strain having no mutations in the endogenous gene having the nucleic acid sequence of SEQ ID NO: 4, within 8 hours after ferulic acid is fed to the mutant strain or the wild-type strain.

A thirteenth embodiment is a mutant strain of the first through the eleventh embodiments, wherein the strain produces at least 200% more vanillin when compared to a wild-type strain having no mutations in the endogenous gene having the nucleic acid sequence of SEQ ID NO: 4, within 8 hours after ferulic acid is fed to the mutant strain or the wild-type strain.

A fourteenth embodiment is a mutant strain of the first through the eleventh embodiments, wherein the strain produces at least 300% more vanillin when compared to a wild-type strain having no mutations in the endogenous gene having the nucleic acid sequence of SEQ ID NO: 4, within 8 hours after ferulic acid is fed to the mutant strain or the wild-type strain.

A fifteenth embodiment is a mutant strain of the first through the eleventh embodiments, wherein the strain produces at least 400% more vanillin when compared to a wild-type strain having no mutations in the endogenous gene having the nucleic acid sequence of SEQ ID NO: 4, within 8 hours after ferulic acid is fed to the mutant strain or the wild-type strain.

A sixteenth embodiment is a mutant strain of the first through the fifteenth embodiments, wherein the mutant strain is obtained by using marker-less CRISPR-based recombinant DNA technology without permanently modifying the mutant strain by introducing any exogenous genetic material.

A seventeenth embodiment is a mutant strain of the first through the fifteenth embodiments, wherein the mutant strain is obtained by contact a strain with the mutagen methyl methanesulfonate.

An eighteenth embodiment is a strain of Amycolaptosis sp., comprising: a non-naturally occurring strain of Amycolaptosis sp., wherein said strain is capable of producing at least 0.5 g vanillin per liter medium within 5 hours after ferulic acid is initially fed to the mutant strain A nineteenth embodiment is a strain of Amycolaptosis sp., comprising: a non-naturally occurring strain of Amycolaptosis sp., wherein said strain is capable of producing at least 0.75 g vanillin per liter medium within 5 hours after ferulic acid is initially fed to the strain.

A twentieth embodiment is a strain of Amycolaptosis sp., comprising: a non-naturally occurring strain of Amycolaptosis sp., wherein said strain is capable of producing at least 0.5 g vanillin per liter medium within 3 hours after ferulic acid is initially fed to the strain.

A twenty-first embodiment is a strain of Amycolaptosis sp., comprising: a non.-naturally occurring strain of Amycolaptosis sp., wherein said mutant strain is capable of producing at least 0.75 g vanillin per liter medium within 3 hours after ferulic acid is initially fed to the strain.

A twenty-second embodiment is a strain of the eighteenth through the twenty-first embodiments, comprising: a mutation in a gene having the nucleic acid sequence of SEQ ID NO: 4, wherein the mutation reduces or eliminates suppression of the ech-fcs operon.

A twenty-third embodiment is a strain of the eighteenth through the twenty-first embodiments, wherein a gene in the strain codes for a protein having a mutation in the amino acid sequence of SEQ ID NO: 5.

A twenty-fourth embodiment is a strain of the eighteenth through the twenty-third embodiments, including a mutation in the vdh gene having a nucleic acid sequence of SEQ IS NO: 6.

A twenty-fifth embodiment is a strain of the eighteenth through the twenty-fourth embodiments, wherein said mutation is a deletion, an insertion, a frameshift mutation, a promoter mutation, a missense mutation, a nonsense mutation, a slicing mutation or a point mutation.

A twenty-sixth embodiment is a strain of the twenty-fifth embodiment, wherein the mutation is a deletion.

A twenty-seventh embodiment is a strain of the twenty-fifth embodiment, wherein the mutation is a frameshift mutation.

A twenty-eighth embodiment is a strain of the twenty-fifth embodiment, wherein the mutation is a promoter mutation.

A twenty-ninth embodiment is a strain of the eighteenth through the twenty-eighth embodiments, wherein the strain is derived from Amycolatopsis sp. accessible under number ATCC 39116.

A thirtieth embodiment is a strain of the eighteenth through the twenty-ninth embodiments, wherein the strain produces at least 100% more vanillin when compared to a wild-type strain having no mutations in the endogenous gene having the nucleic acid sequence of SEQ ID NO: 4, within 8 hours after ferulic acid is fed to the mutant strain or the wild-type strain.

A thirty-first embodiment is a strain of the eighteenth through the twenty-ninth embodiments wherein the strain produces at least 200% more vanillin when compared to a wild-type strain having no mutations in the endogenous gene having the nucleic acid sequence of SEQ ID NO: 4, within 8 hours after ferulic acid is fed to the mutant strain or the wild-type strain.

A thirty-second embodiment is a strain of the eighteenth through the twenty-ninth embodiments, wherein the strain produces at least 300% more vanillin when compared to a wild-type strain having no mutations in the endogenous gene having the nucleic acid sequence of SEQ ID NO: 4, within 8 hours after ferulic acid is fed to the mutant strain or the wild-type strain.

A thirty-third embodiment is a strain of the eighteenth through the twenty-ninth embodiments, wherein the strain produces at least 400% more vanillin when compared to a wild-type strain having no mutations in the endogenous gene having the nucleic acid sequence of SEQ ID NO: 4, within 8 hours after ferulic acid is fed to the mutant strain or the wild-type strain.

A thirty-fourth embodiment is a process for producing vanillin, comprising culturing the strain of any one of the first through the thirty-third embodiments in an appropriate medium comprising a substrate, and recovering the produced vanillin.

A thirty-fifth embodiment is a method for producing vanillin comprising the steps of: culturing a mutant strain of Amycolaptosis sp. according to any one of the first through the seventeenth embodiments, in a medium containing a carbon source; and feeding ferulic acid to the mutant strain for a sufficient period of time to allow conversion of ferulic acid to vanillin.

A thirty-sixth embodiment is a method for producing vanillin comprising the steps of: culturing a strain of Amycolaptosis sp. according to any one of the eighteenth through the thirty-third embodiments, in a medium containing a carbon source; and feeding ferulic acid to the strain for a sufficient period of time to allow conversion of ferulic acid to vanillin.

Other features and advantages of the present invention will become apparent in the following detailed description, taken with reference to the accompanying drawings.

### BRIEF DESCRIPTION OF THE DRAWINGS

For a better understanding of the present disclosure, reference may be made to the accompanying drawings.

FIG. 1. Metabolic pathway for the conversion of ferulic acid to vanillin.

FIG. 2. Early production of vanillin from ferulic acid by six mutant strains of Amycolatopsis sp. and wild type Amycolatopsis sp. (WT). While there was a lag period in the production of vanillin with the wild type Amycolatopsis sp., vanillin production by the six mutant strains according to the present invention showed no lag period for vanillin production.

FIG. 3. Bioconversion of ferulic acid into vanillin by wild-type Amycolatopsis (top) and an exemplary mutant strain of Amycolatopsis cured of the reporter plasmid (bottom).

### BRIEF DESCRIPTION OF THE SEQUENCES

Table 1 briefly describes the sequences disclosed herein and in the attached sequence listing. As known to the skilled artisan, it is noted that prokaryotes use alternate start codons, mailny GUG and UUG, which are translated as formyl-methionine.

TABLE 1

| Sequence Information | |
|---|---|
| SEQ ID NO: 1 | DNA; Nucleic Acid Sequence of promoter region of ech-fcs operon |
| SEQ ID NO: 2 | DNA; Nucleic Acid Sequence of red fluorescent protein |
| SEQ ID NO: 3 | DNA; Nucleic Acid sequence of terminator for reporter gene |
| SEQ ID NO: 4 | DNA; Nucleic acid sequence of echR (repressor of ech-fcs operon) |
| SEQ ID NO: 5 | PRT; Amino Acid Sequence of EchR (repressor of ech-fcs operon) |
| SEQ ID NO: 6 | DNA; Nucleic acid sequence of vdh (vanillin dehydrogenase) |
| SEQ ID NO: 7 | PRT; Amino acid sequence of VDH (Vanillin dehydrogenase) |

### DETAILED DESCRIPTION

As used herein, the singular forms “a,” “an,” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean serving as an example, instance, or illustration. Any embodiment described herein as “exemplary” is not necessarily to be construed as preferred or advantageous over other embodiments.

“Cellular system” is any cells that provide for the expression of ectopic proteins. It includes bacteria, yeast, plant cells and animal cells. It includes both prokaryotic and eukaryotic cells. It also includes the in vitro expression of proteins based on cellular components, such as ribosomes.

“Coding sequence” is to be given its ordinary and customary meaning to a person of ordinary skill in the art and is used without limitation to refer to a DNA sequence that encodes for a specific amino acid sequence.

“Growing” or “cultivating” a cellular system includes providing an appropriate medium that would allow cells to multiply and divide. It also includes providing resources so that cells or cellular components can translate and make recombinant proteins.

The term “complementary” is to be given its ordinary and customary meaning to a person of ordinary skill in the art and is used without limitation to describe the relationship between nucleotide bases that are capable of hybridizing to one another. For example, with respect to DNA, adenosine is complementary to thymine and cytosine is complementary to guanine. Accordingly, the technology of the present invention also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing vanillin.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In one aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art and is used without limitation to refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression,” as used herein, is to be given its ordinary and customary meaning to a person of ordinary skill in the art and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of ordinary skill in the art and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosome. Host organisms containing the transformed nucleic acid fragments are referred to as “transgenic” or “transformed” or “recombinant”.

The terms “transformed,” “transgenic,” and “recombinant,” when used herein in connection with host cells, are to be given their respective ordinary and customary meanings to a person of ordinary skill in the art and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

As defined in the present invention, an organism comprising an exogenous nucleic acid derived from another organism is considered as a genetically modified organism (GMO). If an organism is genetically modified without the introduction of an exogenous nucleic acid molecule, it would be considered a non-genetically modified organism (non-GMO). A non-GMO may have one or more genetic modifications in its endogenous nucleic acid such as a point mutation in the coding sequence of a gene or deletion of an entire coding region of a gene without having any exogenous nucleic acid sequence.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

“Protein expression” refers to protein production that occurs after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein, “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “Best Fit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981: Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% sequence identity, at least about 90% sequence identity, or even greater sequence identity, such as about 98% or about 99% sequence identity. Thus, one embodiment of the invention is a polynucleotide molecule that has at least about 70% sequence identity, at least about 80% sequence identity, at least about 85% sequence identity, at least about 90% sequence identity, or even greater sequence identity, such as about 98% or about 99% sequence identity with a polynucleotide sequence described herein. Polynucleotide molecules that have the activity genes of the current invention are capable of directing the production of vanillin and have a substantial percent sequence identity to the polynucleotide sequences provided herein and are encompassed within the scope of this invention.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following: BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR). In molecular cloning, a vector is a DNA molecule used as a vehicle to artificially carry foreign genetic material into another cell, where it can be replicated and/or expressed (e.g. plasmid, cosmid, Lambda phages). A vector containing foreign DNA is considered recombinant DNA. The four major types of vectors are plasmids, viral vectors, cosmids, and artificial chromosomes. Of these, the most commonly used vectors are plasmids. Common to all engineered vectors are an origin of replication, a multicloning site, and a selectable marker.

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provided are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID RES. 18 6069-74, (1990); Haun et al., BIOTECHNIQUES 13, 515-18 (1992), each of which are incorporated herein by reference).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, and place the coding region in the desired reading frame.

In an embodiment, a polynucleotide for incorporation into an expression vector of the subject technology is prepared using PCR-appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector.

### Fermentative Production of Vanillin

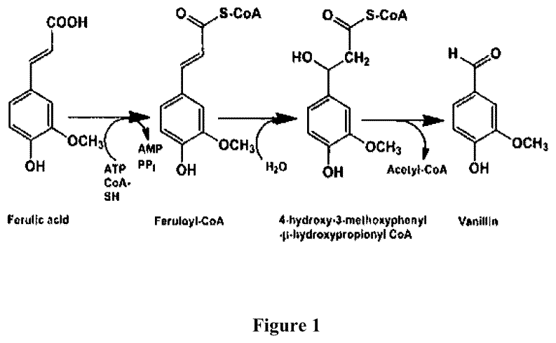

With reference to FIG. 1, ferulic acid metabolism in Amycolatopsis sp. strains goes through a non-β-oxidative deacetylation pathway. The pathway is mediated by two enzymes; namely, feruloyl-coenzyme A (CoA) synthetase (Fcs) encoded by the fcs gene and enoyl-CoA hydratase/aldolase (Ech) encoded by the ech gene. Specifically, the Fcs enzyme mediates the conversion of ferulic acid to feruloyl-CoA, and the Ech enzyme first adds an H₂O to feruloyl-CoA to provide 4-hydroxy-3-methoxyphenyl-β-hydroxypropionyl-CoA, which is then converted into vanillin. Both the ech and fcs genes are within an operon and the expression of these two genes is normally suppressed when Amycolatopsis sp. is grown in a culture medium containing glucose as the source of carbon. Induction of the transcription of these genes requires the addition of ferulic acid or another monomeric lignin precursor such as cinnamic acid, p-coumaric acid, and caffeic acid. As a result, there is a lag period typically from 4 hours to as many as 8 hours after ferulic acid is added to the culture medium before vanillin synthesis could be detected. Such a delay in the production of vanillin and a longer time to complete bioconversion of ferulic acid to vanillin causes a loss of vanillin due to further oxidation by additional enzymes. Hence, there is a need to avoid the initial lag period in vanillin production using ferulic acid. The present invention provides a method to isolate mutants of production strains with the ability to produce vanillin using ferulic acid as a feedstock without an induction period for the expression of the ech and fcs genes.

One way to overcome the lag period associated with vanillin production with ferulic acid in Amycolatopsis sp. is to constitutively express the fcs gene coding for feruloyl-coenzyme A (CoA) synthetase and the ech gene coding for enoyl-CoA hydratase/aldolase under a constitutive promotor. This could be achieved with the construction of plasmid vectors with both the ech and fcs genes under one or more constitutive promoters and transforming the Amycolatopsis sp. cells with such plasmid vectors. To stably maintain the plasmid vector within Amycolatopsis sp. cells, the plasmid vector must either be integrated into the host genome or be maintained within the cell using appropriate antibiotics selection markers and providing corresponding antibiotics in the culture medium. This would require the introduction of exogenous nucleic acids into the Amycolatopsis sp. cells, making such recombinant cells genetically modified organisms (GMOs). The present invention, on the other hand, provides a method to increase the expression of endogenous ech and fcs genes already present within the Amycolatopsis sp. cells without introducing any exogenous nucleic acid molecules into the Amycolatopsis sp. cells. The present invention is based on the idea that the expression of the ech and fcs genes are suppressed by a repressor protein and the inactivation of the gene coding for that repressor protein through an in vivo genetic mutation of such an endogenous gene would relieve the suppression of the transcription of the ech and fcs genes by the repressor protein. The present invention is further based on the expectation that the repressor protein exerts its suppression effect on the ech and fcs gene expression by means of binding to the promoter region of the operon encompassing the ech and fcs genes. Another overarching idea in this present invention is that a reporter gene in a plasmid vector under the control of the promoter of the ech-fcs gene operon would be under the control of the repressor suppressing the transcription of the ech and fcs genes. As a result, when an Amycolatopsis sp. cell is transformed with a plasmid vector carrying a reporter gene under the control of a promoter for the ech-fcs operon and grown in the culture medium with glucose as the source of carbon, there will not be any expression of the reporter gene due to the binding of the repressor protein to the promoter region controlling the transcription of the reporter gene. However, in those cells where there is mutation in the gene coding for the repressor protein, the reporter gene will show its expression in the Amycolatopsis sp. cells. Thus, using the reporter gene under the control of the promoter for the ech-fcs operon, one could screen for the cells with a mutation in the gene coding for the repressor protein that binds the promoter for the ech-fcs operon and suppresses the transcription of the ech and fcs genes.

The reporter gene useful in this present invention is expected to have an easily detectable phenotype. For example, the reporter gene may code for a protein which can convert a substrate into a chromogenic product which can be easily detected. In a preferred embodiment of the present invention, a fluorescent protein such as a green fluorescent protein or a red fluorescent protein is used as a reporter gene in the screening for the Amycolatopsis sp. cells with a mutation in the gene coding for the repressor protein with the ability to bind the promoter region of the ech-fcs operon and suppresses the transcription of the fcs and ech genes.

In one embodiment, the present invention provides a mutagenic protocol involving mutagenic chemicals for increasing the mutagenic frequency for the gene coding for the repressor protein with the ability to bind the promotor region of the ech/fcs operon.

A number of different chemical mutagenic agents are well known in the art. Any one of them can be used in the present invention to increase the frequency of mutations in the gene coding for the repressor protein with the ability to bind the promoter of the ech-fcs operon and suppress their transcription. One class of chemical mutagenic agents is base analogues which are similar to one of the four bases of DNA and the incorporation of such a base analogue would cause a stable mutation. Nitrous oxide, another mutagenic chemical, can convert the amino group of bases into keto group through oxidative deamination. The order of frequency of deamination (removal of amino group) is adenine >cytosine >guanine. Alkylating agents is another group of mutagenic agents useful in adding an alkyl group to the hydrogen bonding oxygen of guanine and adenine residues of DNA. As a result of the alkylation, the likelihood of ionization is increased with the introduction of pairing errors. Some widely used examples of alkylating agents include dimethyl sulphate, ethyl methane sulphonate, ethyl ethane sulphonate and methyl methane sulphonate. Certain dyes such as acridine orange, proflavine and acriflavin which are three ringed molecules of similar dimensions as those of purine pyrimidine pairs can also be used. In aqueous solutions, these dyes can insert themselves in DNA between the bases in adjacent pairs by a process called intercalation. These intercalating agents distort the DNA, resulting in deletion or insertion after replication of the DNA molecule. Due to such deletion or insertion caused by intercalating agents, frameshift mutations can occur. Any of the aforementioned categories of mutagenic agents can be used in the present invention.

In one aspect, mutant strains with the desired phenotype (in this case, vanillin production without a lag period) can be obtained by subjecting spores of a vanillin-producing organism such as Amycolatopsis sp. to chemical mutagens following procedures well known in the art. To help identify the mutant strains with the desired phenotype efficiently, the spores can be first transformed with a plasmid carrying a reporter gene. In a preferred aspect of the present invention, a green or red fluorescent protein is used as a reporter gene under the control of a promoter of the ech-fcs operon. In spores with a repressor that can bind the promoter region of the ech-fcs operon, the expression of that reporter gene will be suppressed (i.e., in the off-state) in a glucose-containing medium and will be turned on only in the presence of ferulic acid. By comparison, in spores with a mutation in the gene coding for the repressor protein, the repressor protein is defective and the reporter gene (e.g., a red fluorescent protein) is expressed even in the absence of ferulic acid. As such, cells exhibiting strong red fluorescence in the absence of ferulic acid are the mutant strains that contain a mutation in the gene coding for the repressor protein that binds the promoter for the ech-fcs operon and have lost the ability to suppress the transcription of the ech and fcs genes. Such mutant strains can be sorted and isolated effectively using a FACS (fluorescence-activated cell sorting) device. Once a mutated spore with the desired phenotype of showing strong red fluorescence in the absence of ferulic acid is isolated, it is grown to a predetermined cell density, then tested to confirm its ability to produce vanillin upon the addition of ferulic acid without exhibiting any lag period.

The selected mutant strain then can be subjected to whole genome sequencing to identify the mutations caused by the chemical mutagenesis process. To confirm that particular mutation(s) in the chromosomal DNA of the selected strains are indeed solely responsible for the observed phenotype of interest, the selected mutant strain is cured of the plasmid carrying the reporter gene using techniques well known in the field of microbial genetics. If the selected strain continues to exhibit the observed phenotype even after curing the reported plasmid vector, it is safe to reach the conclusion that the mutation(s) identified in the selected strain are indeed responsible for the observed phenotype. The role of an identified mutation in inducing the observed phenotype can be further verified by means of introducing the identified mutation into wild-type Amycolatopsis sp. cells and demonstrating that the introduced mutation does confer the desired new phenotype. The introduction of the identified mutation into wild-type Amycolatopsis sp. cells can be carried out using one or more well-known techniques in the field of microbial genetics. In a preferred aspect of the present invention, the introduction of the identified mutation in the repressor protein into wild-type Amycolatopsis sp. cells is carried out using a technique so that no exogenous nucleic acid is introduced into the Amycolatopsis sp. cells for the purpose of maintaining the status of non-GMO.

Genomic sequencing of the mutant strains described above reveals that each of these mutant strains comprises at least one mutation in a gene having the nucleic acid sequence as set forth in SEQ ID NO: 4. This gene was determined to contain an MarR-type transcriptional repressor domain, suggesting it is indeed responsible for negatively regulating the ferulic acid metabolism regulon. Mutation(s) of this gene that result in its activity being significantly reduced or eliminated lead to the relieved repression of the ech-fcs operon, which in turn leads to the expression of the ech and fcs genes without the need to pre-induce with ferulic acid. Accordingly, vanillin production takes place promptly without a lag period. The amino acid sequence translated from the nucleic acid sequence of SEQ ID NO: 4 is set forth as SEQ ID NO: 5.

In some embodiments, the mutation in the echR gene (SEQ ID NO: 4) is a deletion. In some embodiments, the mutation in the echR gene is a frameshift mutation. For example, the frameshift mutation can be after bp 252. In another example, the frameshift mutation can be after bp 166. In some embodiment, the mutation can be a promoter mutation. For example, it can be a promoter mutation G-11A. More generally, the mutations in the echR gene can be selected from the group consisting of a deletion, an insertion, a frameshift mutation, a promoter mutation, a missense mutation, a nonsense mutation, a slicing mutation, and a point mutation, wherein the mutation is capable of causing functional inactivation of the EchR protein, thereby relieving the repression of the ech-fcs operon.

Mutant strains identified according to the present teachings yield more vanillin within a shorter production time compared to a wild-type strain without a mutation in the echR gene. For example, a mutant strain according to the present teachings can produce at least 0.5 g of vanillin per liter of culture medium within 5 hours after ferulic acid is initially fed to the mutant strain. In another embodiment, the mutant strain can produce at least 0.75 g of vanillin per liter of culture medium within 5 hours after ferulic acid is initially fed to the mutant strain. In yet another embodiment, the mutant strain can produce at least 0.5 g of vanillin per liter of culture medium within 3 hours after ferulic acid is initially fed to the mutant strain.

In an embodiment of the present invention, the mutant strain can produce at least 100% more vanillin when compared to a wild-type strain having no mutation in the endogenous echR gene, within 8 hours after ferulic acid is fed to the mutant strain or the wild-type strain. In another embodiment, the mutant strain can produce at least 200% more vanillin when compared to a wild-type strain having no mutation in the endogenous echR gene, within 8 hours after ferulic acid is fed to the mutant strain or the wild-type strain. In yet another embodiment, the mutant strain can produce at least 300% more vanillin when compared to a wild-type strain having no mutation in the endogenous echR gene, within 8 hours after ferulic acid is fed to the mutant strain or the wild-type strain. In a preferred embodiment, the mutant strain can produce at least 400% more vanillin when compared to a wild-type strain having no mutation in the endogenous echR gene, within 8 hours after ferulic acid is fed to the mutant strain or the wild-type strain.

Once a mutant strain of Amycolatopsis sp. is selected with the ability to produce vanillin using ferulic acid without any lag period, the performance of this strain can further be improved, for example, by means of blocking the vanillin degradation pathway that exists within the selected strain. For example, the oxidation of vanillin to vanillic acid by the enzyme vanillin dehydrogenase (Vdh) can be blocked by means of mutating the vdh gene coding for the vanillin dehydrogenase enzyme. Using the techniques well known in the art, one can introduce one or more mutations, including but not limited to a deletion, an insertion, a frameshift mutation, a promoter mutation, a missense mutation, a nonsense mutation, a slicing mutation or a point mutation which are known to functionally inactivate the vanillin dehydrogenase enzyme. In a preferred aspect of the present invention, the mutation of the vdh gene leading to the functional inactivation of vanillin dehydrogenase enzyme is carried out using one or more techniques well known in the art of microbial genetics which would allow the desired mutation in the vdh gene without introducing any exogeneous nucleic acid sequence into the selected mutant strain of Amycolatopsis sp. so that the status of non-GMO is maintained.

It has been reported that enzymes involved in the conversion of benzaldehyde to benzyl alcohol in Escherichia coli are also responsible for the reduction of vanillin to vanillyl alcohol. Deletion of yeaE, dkgA, yqhC, yqhD, yahK and yjgB from E. co/i has eliminated the further reduction of vanillin. One can identify the homologs for these genes in the mutant strains of Amycolatopsis sp. of the present invention having mutation in the promoter region of ech-fcs operon and are useful in the vanillin production using ferulic acid as a feedstock. It is expected that the mutating one of more genes selected from a group consisting of yeaE, dkgA, yqhC, yqhD, yahK and yjgB genes in the echR mutant strains of the present invention would result in the reduction or elimination of vanillic acid production resulting in an increase in vanillin yield.

In a preferred embodiment of the present invention, any further genetic modifications to the mutant Amycolatopsis sp. cells of the present invention can be done using the CRISPAR/CAS system well-known in the field of microbial genetics (see, e.g., US Patent Application Publication 2016/0298096—CRISPR-CAS System, Materials and Methods; Wang et al. (2016), Bacterial Genome Editing with CRISPR-Cas9: Deletion, Integration, Single Nucleotide Modification, and Desirable “Clean” Mutant Selection in Clostridium beijerinckii as an Example, ACS Synth. Biol., DOI: 10.1021/acssynbio.6b00060; Huang et al. (2016), CRISPR interference (CRISPRi) for gene regulation and succinate production in cyanobacterium S. elongatus PCC 7942, Microb Cell Fact, 15:196; Kuivanen et al. (2016), Engineering Aspergillus niger for galactaric acid production: elimination of galactaric acid catabolism by using RNA sequencing and CRISPR/Cas9, Microb Cell Fact, 15:210; Peng et al. (2017), Efficient gene editing in Corynebacterium glutamicum using the CRISPR/Cas9 system, Microb Cell Fact, 16:201; Gorter de Vries et al. (2017), CRISPR-Cas9 mediated gene deletions in lager yeast Saccharomyces pastorianus, Microb Cell Fact, 16:222; Wu et al. (2019), Strategies for Developing CRISPR-Based Gene Editing Methods in Bacteria Small Methods, DOI: 10.1002/smtd.201900560; Ramachandran G and Bikard D (2019), Editing the microbiome the CRISPR way, Phil. Trans. R. Soc. B, 374: 20180103 http://dx.doi.org/10.1098/rstb.2018.0103).

In some embodiments, functional enzymes can be inactivated using antisense RNA technology or RNAi technology (see, e.g., Xu et al. (2018), Antisense RNA: the new favorite in genetic research, Biomed & Biotechnol., 19(10):739-749; Zheng et al (2019), Microbial CRISPRi and CRISPRa Systems for Metabolic Engineering, Biotechnology and Bioprocess Engineering, 24: 579-591). However, in the spirit of the present invention for using non-GMO strains in the bioconversion of ferulic acid to vanillin, it is necessary to make sure that the use of antisense RNA technology and RNAi technology to inactivate a functional protein does not introduce any exogenous nucleic acid into the mutant Amycolatopsis sp. strains selected for the production of vanillin from ferulic acid in a commercial scale and the non-GMO status is maintained.

The culture broth can be prepared and sterilized in a bioreactor. Engineered host strains according to the present invention can then be inoculated into the culture broth to initiate the growth phase. An appropriate duration of the growth phase can be about 5-40 hours, preferably about 10-35 hours and most preferably about 10-20 hours.

After the termination of the growth phase, the substrate ferulic acid can be fed to the culture. A suitable amount of substrate-feed can be 0.1-40 g/L of the fermentation broth, preferably about 0.3-30 g/L. The substrate can be fed as a solid material or as an aqueous solution or suspension. The total amount of substrate can be either fed in one step, in two or more feeding-steps, or continuously.

The bioconversion phase using the Amycolatopsis sp. strains developed in the present invention starts with the beginning of the substrate feed and can last about 5-50 hours, preferably 10-40 hours, and most preferably 15-30 hours, until all substrate is converted to product and by-products. Unlike the wild-type Amycolatopsis sp. cells showing several hours of lag period before vanillin production starts, the Amycolatopsis sp. strains developed in the present invention will be able to produce vanillin right away with the introduction of ferulic acid without showing any lag period.

After the end of the bioconversion phase, the biomass can be separated from the fermentation broth by any well-known methods, such as centrifugation or membrane filtration and the like to obtain a cell-free fermentation broth.

An extractive phase can be added to the fermentation broth using, e.g., a water-immiscible—organic solvent, a plant oil or any solid extractant, e.g., a resin; preferably, a neutral resin. The fermentation broth can be further sterilized or pasteurized. In some embodiments, the fermentation broth can be concentrated. From the fermentation broth, vanillin can be extracted selectively using, for example, a continuous liquid-liquid extraction process, or a batch-wise extraction process.

Advantages of the present invention include, among others, the ability of the Amycolatopsis sp. strains developed in the present invention to start the bioconversion of ferulic acid to vanillin without any lag period to shorten the production period. This highly simplifies the production process, making the process efficient and economical, thus allowing scale-up to industrial production levels.

One skilled in the art will recognize that the vanillin composition produced by the method described herein can be further purified and mixed with fragrant and/or flavored consumable products as described above, as well as with dietary supplements, medical compositions, and cosmeceuticals, for nutrition, as well as in pharmaceutical products.

The disclosure will be more fully understood upon consideration of the following non-limiting examples. It should be understood that these examples, while indicating preferred embodiments of the subject technology, are given by way of illustration only. From the above discussion and these examples, one skilled in the art can ascertain the essential characteristics of the subject technology, and without departing from the spirit and scope thereof, can make various changes and modifications of the subject technology to adapt it to various uses and conditions.

### EXAMPLES

### Bacterial Strains, Plasmids and Culture Conditions.

E. coli strains of DH5α and BL21 (DE3) were purchased from Invitrogen. Plasmid pET28a was purchased from EMD Millipore (Billerica, MA, USA), which was used for gene cloning.

### DNA Manipulation.

All DNA manipulations were performed according to standard procedures. Restriction enzymes, T4 DNA ligase were purchased from New England Biolabs. All PCR reactions were performed with New England Biolabs' Phusion PCR system according to the manufacturer's guidance.

### Example 1

### The Construction of Plasmid with Reporter Gene

Red fluorescent protein (RFP) was used as a reporter gene in the present invention. The promoter for ech-fcs operon, comprising 305 bp immediately before the ech gene, was PCR-amplified from the Amycolatopsis sp. ATCC 39116 genome. This fragment was then cloned into the pRLE6 vector along with a codon-optimized red fluorescent protein (RFP) variant to create plasmid MPL210.

### Example 2

### Chemical Mutagenesis and Selection of Mutant Strains

Single-celled spores of Amycolatopsis sp. ATCC 39116 containing the MPL210 reporter plasmid were treated with the mutagen methyl methanesulfonate. The mutagenized spores were flowed through a flow cytometer, and the brightest red cells were sorted out and plated on a medium lacking ferulic acid. Colonies were visually scanned for red color, and six mutant colonies were selected for further analysis.

### Example 3

### Assaying Mutant Strains for Bioconversion of Ferulic Acid to Vanillin

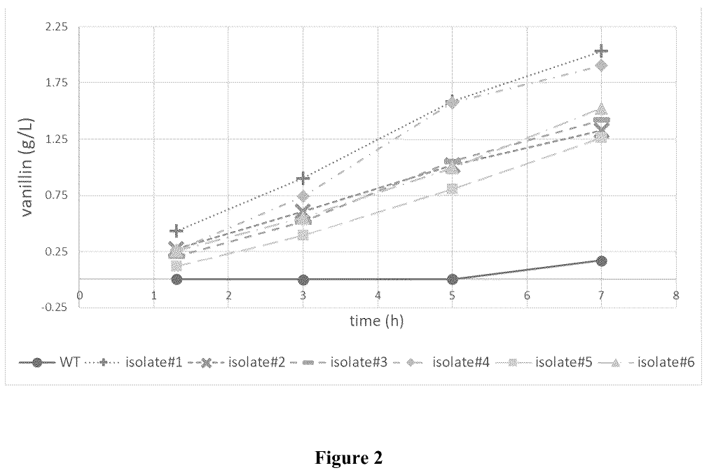

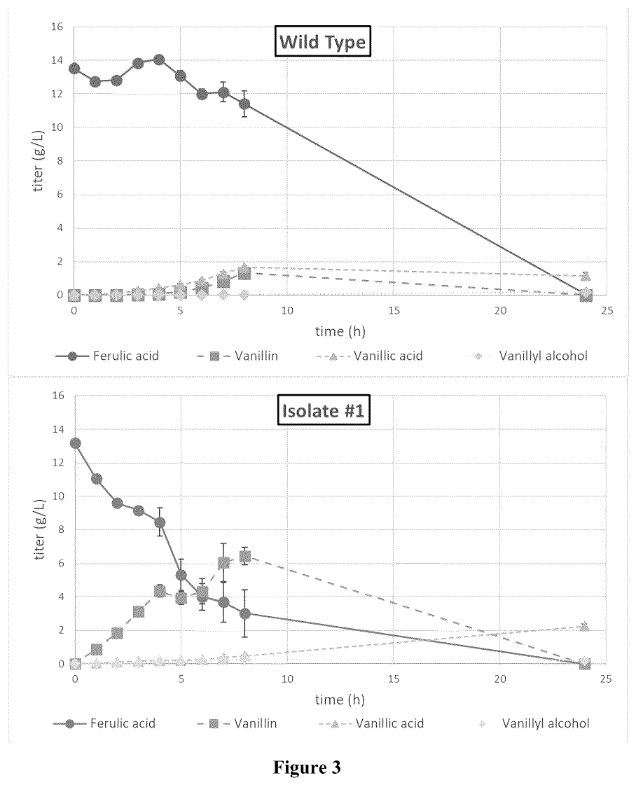

Six mutant colonies were selected and tested for bioconversion of ferulic acid to vanillin (FIG. 2). All six mutant strains began the conversion of ferulic acid to vanillin immediately, without the typical lag period displayed by the wild-type strain. The plasmid with the reporter gene from these mutant strains was cured using the technique well known in the art to restore the non-GMO status for these mutant strains. These cured mutant strains were assayed for the bioconversion of ferulic acid to vanillin and all of the cured mutant strains were found to retain the phenotype of bioconversion of ferulic acid to vanillin without any lag period (FIG. 3), indicating that the mutation causing the desired bioconversion phenotype resides in the chromosomal DNA of the mutant strains and not in the plasmid carrying the reporter gene. There was also very little accumulation of vanillic acid during the early stages of bioconversion, as opposed to the wild-type strain that produced more vanillic acid than vanillin (FIG. 3).

After an eight-hour incubation with ferulic acid at the concentration of 13 g/L, one of the mutant strains produced 6.43 g/L of vanillin which is 4.87 times more vanillin production when compared to the bioconversion of ferulic acid to vanillin by wild-type Amycolatopsis sp. (1.32 g/L vanillin). It is also important to note here that the during the incubation period of 8 hours, the mutant strain showed a reduced accumulation of vanillic acid (0.497 g/L) when compared the vanillic acid accumulation of 1.67 g/L in the wild-type strain of Amycolatopsis sp.

### Example 4

### Identification of Mutant Gene

Whole genome sequencing was followed with two mutant strains to identify the mutation causing the observed phenotype of bioconversion of ferulic acid to vanillin without any lag period. Genome sequencing of two mutant strains revealed that the mutation is in one particular gene: locus tag AMY39116_RS0323770 (Table 1). The same gene was also found mutated in all six mutant strains selected from chemical mutagenesis (Table 2). This mutated gene contains a MarR-type transcriptional repressor domain, suggesting it is indeed responsible for negatively regulating the ferulic acid metabolism by means of binding to the promoter region of ech-fcs operon. This gene showing one or more mutations in all six mutant strains is referred in this application as echR.

TABLE 2
Genotype of the strains from
chemical mutagenesis of Amycolatopsis sp.
ATCC 39116

| Isolate # | Mutation in echR allele |
|---|---|
| 1 | Deletion |
| 2 | Deletion |
| 3 | Frameshift after bp 252 |
| 4 | Frameshift after bp 166 |
| 5 | Promoter mutation G-11A |
| 6 | Deletion |

### Example 5

### HPLC Analysis

The HPLC analysis of vanillin was carried out with a Vanquish Ultimate 3000 system. The compounds were identified by their retention times, as well as the corresponding spectra, which were identified with a diode array detector in the system.

### Example 6

### Bioconversion of Ferulic Acid to Vanillin

Wild-type and mutant strains of Amycolatopsis sp. were grown to saturation in 10 mL seed medium (yeast extract 12 g/L, glucose 10 g/L, MgSO₄ 0.2 g/L, K₂HPO₄ 7.5 g/L, KH₂PO₄ lg/L, pH 7.2) for 24 hours at 37° C., diluted 1:20 dilution into 10 mL conversion medium (yeast extract 5 g/L, glucose 8 g/L, malt extract 10 g/L, MgSO₄ 0.2 g/L) for 24 hours at 37° C. and fed with ferulic acid as a substrate. Samples were taken at the indicated times by collection of the bacterial cultures into methanol for HPLC analysis.

Referring to FIG. 2, while the wild-type strain did not begin production of vanillin until after the 5-hour point, each of the six mutant strains selected according to the present invention showed detectable levels of vanillin almost immediately, and at the 3-hour point, five of the six mutant strains has produced at least ˜0.5 g/L of vanillin, and at the 5-hour point, each of the six mutant strains has produced at least 0.75 g/L of vanillin. At the 7-hour point, each of the six mutant strains has produced at least 1.25 g/L of vanillin, whereas the wild-type strain has produced only ˜0.2 g/L of vanillin.

Referring to FIG. 3, it can be seen that over 24 hours of conversion time, the mutant strain according to the present invention was able to produce >6 g/L of vanillin in 8 hours after ferulic acid was added. By comparison, the wild-type strain was able to produce <2 g/L of vanillin in 8 hours after ferulic acid was added. In addition, with the wild-type strain, it is noted that there is also a relative higher degradation of vanillin to vanillic acid. For example, at the 8-hour point, the wild-type strain has accumulated ˜2 g/L of vanillic acid, while the mutant strain has accumulated less than 0.5 g/L of vanillic acid.

This application refers to various issued patents, published patent applications, journal articles, and other publications, all of which are incorporated herein by reference. All references, patents and patent applications disclosed herein are incorporated by reference with respect to the subject matter for which each is cited, which in some cases may encompass the entirety of the document. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above description, but rather is as set forth in the appended claims. Those of ordinary skill in the art will appreciate that various changes and modifications to this description may be made without departing from the spirit or scope of the present disclosure, as defined in the following claims.

1.-36. (canceled)

37. A non-GMO mutant strain of Amycolaptosis sp., wherein said mutant strain is capable of producing at least 0.5 g vanillin per liter medium within 5 hours after ferulic acid is initially fed to the mutant strain.

38. The mutant strain of claim 37, wherein said mutant strain is capable of producing at least 0.75 g vanillin per liter medium within 5 hours after ferulic acid is initially fed to the mutant strain.

39. The mutant strain of claim 37, wherein said mutant strain is capable of producing at least 0.5 g vanillin per liter medium within 3 hours after ferulic acid is initially fed to the mutant strain.

40. The mutant strain of claim 37, said mutant strain comprising a mutation in an endogenous gene having the nucleic acid sequence of SEQ ID NO: 4, wherein the mutation reduces or eliminates suppression of ech-fcs operon.

41. The mutant strain of claim 37, wherein the strain encodes an endogenous protein having a mutation in the amino acid sequence of SEQ ID NO: 5.

42. The mutant strain of claim 37, further including a mutation in the vdh gene having a nucleic acid sequence of SEQ ID NO: 6.

43. The mutant strain of claim 40, wherein said mutation is a deletion, an insertion, a frameshift mutation, a promoter mutation, a missense mutation, a nonsense mutation, a slicing mutation or a point mutation.

44. The mutant strain of claim 43, wherein the mutation is a deletion.

45. The mutant strain of claim 43, wherein the mutation is a frameshift mutation.

46. The mutant strain of claim 43, wherein the mutation is a promoter mutation.

47. The mutant strain of claim 37, wherein the mutant strain is a mutant of the strain Amycolatopsis sp. accessible under number ATCC 39116.

48. The mutant strain of claim 37, wherein the mutant strain produces at least 100% more vanillin when compared to a wild-type strain having no mutations in the endogenous gene having the nucleic acid sequence of SEQ ID NO: 4, within 8 hours after ferulic acid is fed to the mutant strain or the wild-type strain.

49. The mutant strain of claim 37, wherein the mutant strain produces at least 200% more vanillin when compared to a wild-type strain having no mutations in the endogenous gene having the nucleic acid sequence of SEQ ID NO: 4, within 8 hours after ferulic acid is fed to the mutant strain or the wild-type strain.

50. The mutant strain of claim 37, wherein the mutant strain produces at least 300% more vanillin when compared to a wild-type strain having no mutations in the endogenous gene having the nucleic acid sequence of SEQ ID NO: 4, within 8 hours after ferulic acid is fed to the mutant strain or the wild-type strain.

51. The mutant strain of claim 37, wherein the mutant strain produces at least 400% more vanillin when compared to a wild-type strain having no mutations in the endogenous gene having the nucleic acid sequence of SEQ ID NO: 4, within 8 hours after ferulic acid is fed to the mutant strain or the wild-type strain.

52. The mutant strain of claim 37, wherein the mutant strain is obtained by using marker-less CRISPR-based recombinant DNA technology without permanently modifying the mutant strain by introducing any exogenous genetic material.

53. The mutant strain of claim 37, wherein the mutant strain is obtained by contacting a strain with the mutagen methyl methanesulfonate.

54. A strain of Amycolaptosis sp., comprising: a occurring strain of Amycolaptosis sp., wherein said strain is capable of producing at least 0.5 g vanillin per liter medium within 5 hours after ferulic acid is initially fed to the mutant strain.

55. A strain of Amycolaptosis sp., comprising: a strain of Amycolaptosis sp., wherein said strain is capable of producing at least 0.75 g vanillin per liter medium within 5 hours after ferulic acid is initially fed to the strain.

56. A strain of Amycolaptosis sp., comprising: a strain of Amycolaptosis sp., wherein said strain is capable of producing at least 0.5 g vanillin per liter medium within 3 hours after ferulic acid is initially fed to the strain.

57. A strain of Amycolaptosis sp., comprising: a strain of Amycolaptosis sp., wherein said strain is capable of producing at least 0.75 g vanillin per liter medium within 3 hours after ferulic acid is initially fed to the strain

58. The strain of claim 54, said strain comprising a mutation in a gene having the nucleic acid sequence of SEQ ID NO: 4, wherein the mutation reduces or eliminates suppression of the ech-fcs operon.

59. The strain of claim 54, wherein a gene in the strain codes for a protein having a mutation in the amino acid sequence of SEQ ID NO: 5.

60. The strain of claim 54, further including a mutation in the vdh gene having a nucleic acid sequence of SEQ ID NO: 6.

61. The strain of claim 58, wherein said mutation is a deletion, an insertion, a frameshift mutation, a promoter mutation, a missense mutation, a nonsense mutation, a slicing mutation or a point mutation.

62. The strain of claim 61, wherein the mutation is a deletion.

63. The strain of claim 61, wherein the mutation is a frameshift mutation.

64. The strain of claim 61, wherein the mutation is a promoter mutation.

65. The strain of claim 54, wherein the strain is derived from Amycolatopsis sp. accessible under number ATCC 39116.

66. The strain of claim 54, wherein the strain produces at least 100% more vanillin when compared to a wild-type strain having no mutations in the endogenous gene having the nucleic acid sequence of SEQ ID NO: 4, within 8 hours after ferulic acid is fed to the mutant strain or the wild-type strain.

67. The strain of claim 54, wherein the strain produces at least 200% more vanillin when compared to a wild-type strain having no mutations in the endogenous gene having the nucleic acid sequence of SEQ ID NO: 4, within 8 hours after ferulic acid is fed to the mutant strain or the wild-type strain.

68. The strain of claim 54, wherein the strain produces at least 300% more vanillin when compared to a wild-type strain having no mutations in the endogenous gene having the nucleic acid sequence of SEQ ID NO: 4, within 8 hours after ferulic acid is fed to the mutant strain or the wild-type strain.

69. The strain of claim 54, wherein the strain produces at least 400% more vanillin when compared to a wild-type strain having no mutations in the endogenous gene having the nucleic acid sequence of SEQ ID NO: 4, within 8 hours after ferulic acid is fed to the mutant strain or the wild-type strain.

70. A process for producing vanillin, comprising culturing the mutant strain of claim 37 in an appropriate medium comprising a substrate, and recovering the produced vanillin.

71. A method for producing vanillin comprising the steps of:
a. culturing a mutant strain of Amycolaptosis sp. according to claim 37 in a medium containing a carbon source; and
b. feeding ferulic acid to the mutant strain for a sufficient period of time to allow conversion of ferulic acid to vanillin.

72. A method for producing vanillin comprising the steps of:
a. culturing a strain of Amycolaptosis sp. according to claim 54 in a medium containing a carbon source; and
b. feeding ferulic acid to the strain for a sufficient period of time to allow conversion of ferulic acid to vanillin.


SEQUENCE LISTING (7 sequences)

SEQ ID NO: 1 - DNA, 305 bp, Amycolatopsis sp. ATCC 39116
  misc_feature (1)..(305): Nucleic Acid Sequence of promoter region of ech-fcs operon
gcccccgattgtccgcaccggccgcggccgccgggaaggcgccgcccggtcgcggcaaccgcgctcaccggcgcgtcgcgcccgagcccgtcaccctcagtccgctggaggcgcgggcggcggtgcagtcagcgccgaacggcctgttcaccgggctcggcggttcctgtccgaggcgctgctgctggccgcgctcggcgggctggtcgccgccgtcctgatcggcgcgccggccatctgaccttgacgccgtcggcccgctcttgctatccctatatcagaactactgatatagggagcgatgc

SEQ ID NO: 2 - DNA, 678 bp, Artificial
  Synthetic polynucleotide
  misc_feature (1)..(678): Nucleic Acid Sequence of red fluorescent protein - codon optimized for Amycolatopsis
  misc_feature (676)..(678): stop codon
atggccagcagcgaggatgtgatcaaggagtttatgcgcttcaaggtgcgcatggaggggtccgtgaacggccacgagttcgagatcgagggtgagggcgagggccgcccgtacgaggggacccagaccgcaaagctgaaggtcaccaagggcggccccctgcccttcgcgtgggacatcctgagcccacagttccagtacggctccaaggcgtacgtcaagcacccggcggacatccccgactacctgaagctgagcttcccggagggcttcaagtgggagcgggtcatgaacttcgaggacgggggcgtggtcacggtgacgcaggacagctcgttgcaggatggcgagttcatctacaaggtgaagctgcgcggcacgaacttccccagcgacggccccgtgatgcagaaaaagacgatgggctgggaggcgtccaccgagaggatgtacccggaggacggcgccctgaagggcgagatcaagatgcggctgaaactgaaggacggaggccactacgacgcggaggtcaagacgacgtacatggcgaagaagcccgtgcagcttccgggcgcatacaagacggacatcaagctggacatcacgtcccacaacgaggactatacgatcgtcgagcagtacgagcgggcggagggccgccactccacgggcgcgtga

SEQ ID NO: 3 - DNA, 137 bp, Artificial
  Synthetic polynucleotide
  misc_feature (1)..(137): Nucleic Acid sequence of terminator for reporter gene
aggtccaggcatcaaataaaacgaaaggctcagtcgaaagactgggcctttcgttttatctgttgtttgtcggtgaacgctctctactagagtcacactggctcaccttcgggtgggcctttctgcgtttatagctt

SEQ ID NO: 4 - DNA, 483 bp, Amycolatopsis sp. ATCC 39116
  misc_feature (1)..(483): Nucleic acid sequence of echR (repressor of ech-fcs operon)
  misc_feature (481)..(483): stop codon
gtggtgaccgaatcccgcgccgaggacgccccgctgaccctctacctggtcaagcggctggagctggtgatccgctcgctgatggacgacgcgctgcgcccgttcgggctgaccaccctgcagtacaccgcgctgaccgcgctgcggcaccgcaacgggctgtcgtccgcgcagctcgcgcgccgctcgttcgtccggccccagaccatgcacaccatggtgctcacgctggagaagtacgggctcatcgagcgcgcggaggacccggccaaccgccgggtcctgctcgccaccctcaccgagcgcggcaagcaggtcctcgacgagtgcacgccgctggtccgggagctcgaagaccggatgctctccggcatggacgacgaccgccgcgccgggttccgccgggacctggaggacggctacggcatgctcgcctcgcacgccaacgctcagcgcgcgttgacgaacggcggcggcgagtaa

SEQ ID NO: 5 - protein, 160 aa, Amycolatopsis sp. ATCC 39116
  MISC_FEATURE (1)..(160): Amino Acid Sequence of EchR (repressor of ech-fcs operon)
MVTESRAEDAPLTLYLVKRLELVIRSLMDDALRPFGLTTLQYTALTALRHRNGLSSAQLARRSFVRPQTMHTMVLTLEKYGLIERAEDPANRRVLLATLTERGKQVLDECTPLVRELEDRMLSGMDDDRRAGFRRDLEDGYGMLASHANAQRALTNGGGE

SEQ ID NO: 6 - DNA, 1461 bp, Amycolatopsis sp. ATCC 39116
  misc_feature (1)..(1461): Nucleic acid sequence of vdh (Vanillin dehydrogenase)
  misc_feature (1459)..(1461): stop codon
gtgagctttctcgacgacgagaagtggaccggacgcgtcttcaccggcagctgggagcgcgcggcgggcggcgacgcggccgtcatcgagcccgcgaccggcgacgaactggggcgcgtcggcatcgcctcgccccaggacctggcggcctccgcggccaaggcggccgaggcgcagcgcgcctgggcggcgacctccttccaagaacgcgccgcggtcctgcgccgcgccggcgacctgtggcagcagcacgccgccgagctgaaggactggctgatccgcgagtcgggcagcatccccggcaaggccgacttcgaactgcacgtcgccgcgcaggagtgctacgaggccgccgcgctgccctcccacccgacgggtgaggtcctgccgagcgaggcgccgcggctgagcatggcccgccgcgtgcccgccggcgtggtcggcgtgatcgcgccgttcaacgcgccgctgatcctgtcgatccgctcggtcgcgccggcgctggcgctgggcaacagcgtcgtgctcaagccggacccccgcaccgcggtctgcggtggcgtggcgctggccagggtcttcgaggaggccgggctgcccgccggggtcctgcacgtgctgccgggcggcccggacgtcggcgccgcgctggtcgaggacaagcacgtccgcgtcatctcgttcaccggatcgaccgccgcgggccgcgcggtcggcgagtccgcgggccgccacctcaagcgcgcccacctggaactgggcggcaactcggcgctgatcgtgctcgacgacgccgacctggagcaggcgatgagcgccgccgcgtggggctcgttcttccaccagggccagatctgcatgaccaccgggcggcacctggtgcacgcctcactctacgacgaatacgtggaccgcctggcggacaaggccagccacctgccggtgggcaacccgttcaccgagcaggtcgcgctcggcccgatcatcgacgccaagcagcgcgacaagatccacggcctggtgacgtccagtgtggacgccggcgcgaaggtcgccgcgggcggcacctacgaggacctcttctaccgcgccaccgtgctcgccggcgcgggcccctcggtgcccgcctacgaccaggaggtgttcggcccggtcgccccggtcgcgaagttcaccagcctggacgaggccgcgaagctcgcgtcggagagcgagtacgggctgtcgctgggcatcatcaccgcggacgtggcgaagggactggcgctggccgaccgcatcccgaccggcatcgcgcacatcaacgaccagacggtcaacgacgaggcgctggccccgttcggcggcgtgttcgactccggcaccggctcccgcttcggcgggccggccgcgaacatcgaggcgttcaccgagacccgctgggtcacgatgcgcggcgacgtcgccggctacccgttctga

SEQ ID NO: 7 - protein, 486 aa, Amycolatopsis sp. ATCC 39116
  MISC_FEATURE (1)..(486): Amino acid sequence of Vdh (Vanillin dehydrogenase) protein
VSFLDDEKWTGRVFTGSWERAAGGDAAVIEPATGDELGRVGIASPQDLAASAAKAAEAQRAWAATSFQERAAVLRRAGDLWQQHAAELKDWLIRESGSIPGKADFELHVAAQECYEAAALPSHPTGEVLPSEAPRLSMARRVPAGVVGVIAPFNAPLILSIRSVAPALALGNSVVLKPDPRTAVCGGVALARVFEEAGLPAGVLHVLPGGPDVGAALVEDKHVRVISFTGSTAAGRAVGESAGRHLKRAHLELGGNSALIVLDDADLEQAMSAAAWGSFFHQGQICMTTGRHLVHASLYDEYVDRLADKASHLPVGNPFTEQVALGPIIDAKQRDKIHGLVTSSVDAGAKVAAGGTYEDLFYRATVLAGAGPSVPAYDQEVFGPVAPVAKFTSLDEAAKLASESEYGLSLGIITADVAKGLALADRIPTGIAHINDQTVNDEALAPFGGVFDSGTGSRFGGPAANIEAFTETRWVTMRGDVAGYPF